SEQUENCE LISTING (2 sequences)

SEQ ID NO: 1 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 2 - DNA, 1590 bp, Listeria monocytogenes
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataa

(19) United States
(12) Patent Application Publication
(10) Pub. No.: US 2023/0190906 A1
(43) Pub. Date: Jun. 22, 2023

(54) NON-TOXIC LISTERIOLYSIN O POLYPEPTIDES AND USES THEREOF

(71) Applicant: Ohio State Innovation Foundation, Columbus, OH (US)

(72) Inventor: Stephanie SEVEAU, Dublin, OH (US)

(21) Appl. No.: 17/765,428

(22) PCT Filed: Oct. 1, 2020
(86) PCT No.: PCT/US2020/053707

§ 371 Date: Mar. 30, 2022

Related U.S. Application Data
(60) Provisional application No. 62/908,877, filed on Oct. 1, 2019.

(51) Int. Cl. A61K 39/02; C07K 14/195; A61K 39/39; A61P 31/04

(52) U.S. Cl. CPC A61K 39/02; A61K 39/39; A61P 31/04; C07K 14/195; A61K 2039/55544

(57) ABSTRACT

The present disclosure relates to listeriolysin O polypeptides and vaccine compositions for treating and preventing Listeria infection.

### CROSS-REFERENCE TO RELATED APPLICATIONS

This application claims the benefit of priority to U.S. Provisional Application No. 62/908,877 filed Oct. 1, 2019, the disclosure of which is incorporated herein by reference in its entirety.

### STATEMENT REGARDING FEDERALLY SPONSORED RESEARCH

This invention was made with government support under grant number R01AI107250 awarded by the National Institutes of Health. The government has certain rights in the invention.

### FIELD

The present disclosure relates to non-toxic listeriolysin O polypeptides and vaccine compositions, and uses thereof.

### BACKGROUND

Listeria monocytogenes is a foodborne pathogen and the causative agent of the life-threatening disease listeriosis. The risk and severity of listeriosis are significantly increased among pregnant women, the elderly, infants, and individuals with a compromised immune system. Listeriosis clinical manifestations include septicemia, meningitis, encephalitis, miscarriage, stillbirth and severe infection of neonates with an associated mortality rate ranging from 16-25% despite treatment. Although the food industry has rigorous standards for prevention and surveillance of Listeria contamination, the reported number of listeriosis cases in the US more than doubled from 2007-2014. With increasing incidence of listeriosis and its associated high fatality rate, a vaccine targeting L. monocytogenes can offer an effective preventative measure to reduce the risk of this deadly disease in susceptible populations such as pregnant women and the elderly. In particular, the aging population representing approximately 80% of listeriosis patients is constantly increasing. Therefore, what is needed is a vaccine for preventing or treating L. monocytogenes infection.

### SUMMARY

Disclosed herein are non-toxic listeriolysin O polypeptides and vaccine compositions, and methods of use thereof. Disclosed herein is the generation of a full-length LLO toxoid (LLO^{T}) in which the Thr-Leu (T515G/L516G) cholesterol recognition motif in domain 4 was substituted with two glycine residues. Using LLO^{T} and adjuvant, a novel vaccine was created that protects against infection by L. monocytogenes. This vaccine elicits CD4⁺ Th1 and CD8⁺ cells producing IFN-y and B cells producing LLO-neutralizing antibodies. The advantages of developing a LLO^{T}-based subunit vaccine are safety, the fact that LLO^{T} binds antigen-presenting cells and contains all native antigens for efficient activation of T and B cell responses, while LLO toxicity is abrogated. Finally, this vaccine elicited a response that neutralizes LLO, which is the most critical virulence factor of the bacterium.

In some aspects, disclosed herein is a polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

In some embodiments, the amino acid substitution is at amino acid position 515. In some embodiments, the amino acid substitution is at amino acid position 516. In some embodiments, the non-toxic listeriolysin O comprises amino acid substitutions at amino acid positions 515 and 516.

In some embodiments, the amino acid substitution at amino acid position 515 is selected from the group consisting of T515G and T515A. In some embodiments, the amino acid substitution at amino acid position 516 is selected from the group consisting of L516G and L516A. In some embodiments, the non-toxic listeriolysin O binds to a cell membrane. In some embodiments, the non-toxic listeriolysin O binds to an antigen-presenting cell.

In some aspects, disclosed herein is a nucleic acid comprising a genetically modified listeriolysin O gene comprising one or more point mutations, wherein the genetically modified listeriolysin O gene encodes a polypeptide of any preceding aspect.

In some aspects, disclosed herein is a recombinant DNA vector comprising the nucleic acid of any preceding aspect.

In some aspects, disclosed herein is a vaccine comprising: a non-toxic listeriolysin O comprising an amino acid substitution at one or more amino acid positions when compared to SEQ ID NO: 1, wherein the one or more amino acid positions are selected from the group consisting of 515 and 516.

In some embodiments, the vaccine further comprises one or more adjuvants. In some embodiments, the one or more adjuvants are selected from the group consisting of cholera toxin including the β subunit of cholera toxin (CTB), and other detoxified derivatives of cholera toxin. Additional adjuvants can include Freund’s incomplete adjuvant, Freund’s Complete adjuvant, monophosphoryl lipid A, QS-21, salts, i.e., AlK(SO4)2, AlNa(SO4)2, AlNH4(SO4)2, silica, kaolin, carbon polynucleotides, i.e., poly IC and poly AU. Still other adjuvants can include QuilA, Alhydrogel, and the like. Optionally, the vaccine contemplated herein can be combined with immunomodulators that stimulate Toll-like receptors (such as poly(I:C) and CpG motifs) and cytosolic immune sensor (such as cyclic di-nucleotides such as c-di-AMP, c-di-GMP and the like; bacterial mRNA) and immunostimulants (such as interleukins, interferons and the like).

In some aspects, disclosed herein is a method of preventing a Listeria infection, comprising administering to a subject an effective amount of a vaccine comprising: a polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

In some embodiments, administering the vaccine activates CD4+ Th1s, CD8 T cells, and B cells.

In some embodiments, the Listeria is Listeria monocytogenes. In some embodiments, the subject is a human.

### BRIEF DESCRIPTION OF THE DRAWINGS

The accompanying figures, which are incorporated in and constitute a part of this specification, illustrate several aspects described below. In the text below, the listeriolysin O toxoid is referred to as LLO^{T}.

FIGS. 1A-1C. LLO^{T} does not bind to cholesterol. (FIG. 1A) Recombinant LLO, LLO^{T}, LLO W492A, and LLO D1-3 (1 µg loaded) were subjected to SDS-PAGE and stained with Coomassie blue. (FIG. 1B) Representative CD spectra of LLO and LLO^{T} (0.5 mg/ml). (FIG. 1C) LLO and LLO^{T} were incubated on a PVDF membrane pre-coated with a serial dilution of a cholesterol. LLO and LLO^{T} binding to cholesterol was visualized by dot blot.

FIGS. 2A-2D. LLO^{T} binds to host cell membranes. Control HeLa cells (FIG. 2A) and Hela cells treated with 5 mM methyl-β-cyclodextrin (mβCD) (FIG. 2B) were exposed to LLO or LLO^{T} for 10 min at 4° C. (FIG. 2C) HeLa cells pre-treated, or not, with mβCD were exposed to LLO D1-3 for 10 min at 4° C. (FIG. 2D) THP-1 cells were exposed to LLO or LLO^{T} for 10 min at 4° C. (A, B, C, D) After incubation at 4° C. with the various toxin forms, cells were washed, lysed, and subjected to western blot analysis using anti-LLO and anti-actin antibodies. Representative western blots were selected from at least 3 independent experiments.

FIG. 3. LLO^{T} is non- hemolytic. The EC₅₀ of LLO, LLO W492A, LLO^{T}, and LLO D1-3 was measured from four independent experiments, each performed in duplicate. P values were calculated using a two-tailed Student’s t-test (* = P, <0.05; ** = P <0.01; *** = P <0.001).

FIGS. 4A-4B. Immunization with LLO^{T} and Cholera Toxin protects mice against infection by L. monocytogenes. Mice were immunized at weekly intervals, for 3 consecutive weeks, by intraperitoneal injection of PBS (negative control), cholera toxin adjuvant (CT, 1 mg), LLO^{T} (20 mg), or LLO^{T} (20 mg) plus cholera toxin (1 mg) (LLO^{T}+ CT). At day 28, mice were intravenously inoculated with 2 x 10⁴ L. monocytogenes and sacrificed after 72 h to collect blood and enumerate bacterial colony forming units (CFUs) were enumerated in the liver (FIG. 4A) and spleen (FIG. 4B). Results are expressed as CFUs/organ and medians are presented. Data are from 3 independent experiments, with a total of 4 male mice and 14 female mice per experimental condition (13 female in LLO^{T} + CT). Statistical significance was calculated using a two-sided Mann-Whitney test, ** P < 0.01.

FIGS. 5A-5B. Immunization with LLO^{T} and Alum does not protect mice against infection by L. monocytogenes. Mice were immunized at weekly intervals, for 3 consecutive weeks, by intraperitoneal injection of PBS (negative control), LLO^{T} (20 mg), Alum (40 µg), or LLO^{T} (20 mg) plus alum (40 µg). At day 28, mice were intravenously inoculated with 2 x 10⁴ L. monocytogenes and sacrificed after 72 h to collect blood and enumerate bacterial colony forming units (CFUs) in the liver (FIG. 5A) and spleen (FIG. 5B). Data are from 1 experiment, including 4 male plus 4 female/experimental condition. Results are expressed as CFUs/organ and medians are presented. Statistical significance was calculated using a two-sided Mann-Whitney test, N.S. = Not statistically significant.

FIG. 6. LLO^{T}-specific IgG production in mice immunized with LLO^{T} and adjuvants. The titers of LLO^{T}-specific IgG, IgG1 and IgG2a, IgG2b, and IgG3 were determined by ELISA in serially diluted (1:2) sera from mice immunized with LLO^{T} alone, LLO^{T}+CT or LLO^{T}+Alum. Serum dilution antibody titers were determined as the last dilutions of sera that gave an absorbance > 0.1 above that of control sera from naive mice. Results are expressed as Log2 values of serum dilution titers. Statistical significance was calculated using a one-way ANOVA, * P < 0.05, ** P < 0.01. N = titers from 8 mice for each group.

FIG. 7. Immunization with LLO and cholera toxin generates LLO-neutralizing antibodies. IgGs (15 µg/ml) were purified from pooled sera isolated from mice immunized with PBS, LLO^{T} + CT or LLO^{T} + Alum and tested for their ability to inhibit LLO hemolytic activity. As negative and positive controls, erythrocytes were incubated with PBS or Triton X-100, respectively. Data are representative of 4 independent experiments.

FIGS. 8A-8E. Analysis of T cell responses in the different groups. Spleens were isolated and homogenized into a cell suspension and cultured for 5 days in the presence of 5 µg/ml LLO^{T}. The frequencies of LLO^{T}-specific Th1 (CD3⁺CD4⁺IFN-γ⁺ and CD3⁺CD4⁺TNF-α⁺) (FIG. 8A); Th2 (CD3⁺CD4⁺IL-5⁺, CD3⁺CD4⁺IL-4⁺, and CD3⁺CD4⁺IL-10⁺) (FIG. 8B and FIG. 8C); Th17 (CD3⁺CD4⁺IL-17A⁺) (FIG. 8D); and Tfh (CD3⁺CD4⁺IL-21⁺) (FIG. 8E) were determined by flow cytometry. Data were expressed as mean % positive cells ± standard deviation among the CD3⁺CD4⁺ cells. Statistical differences were determined by one-way ANOVA and significant differences were considered at (* p ≤ 0.05). Data are from 2 mice for PSB and LLO^{T} and from 3 mice for all other groups.

FIGS. 9A-9B. Immunization with LLO^{T} and cholera toxin is protective in µMT^{-/-} mice that lack mature B cells. WT mice and µMT^{-/-} mice (4 male and 4 female mice/experimental condition with the exception of the LLO^{T} condition where 4 female and 3 male mice are shown) were immunized at weekly intervals for 3 consecutive weeks by intraperitoneal injection of PBS (negative control), LLO^{T} (20 mg), LLO^{T} (20 mg) plus cholera toxin (1 mg), or LLO^{T} (20 mg) plus alum (40 µg). At day 28, mice were intravenously inoculated with 2 x 10⁴ L. monocytogenes and sacrificed after 72 h to enumerate bacterial colony forming units (CFUs) in the liver (FIG. 9A) and spleen (FIG. 9B). Results are expressed as CFUs/organ and medians are presented. Statistical significance was calculated using a two-sided Mann-Whitney test, * P <0.05, ** P < 0.01.

FIG. 10. Profile of antigen-specific CD3+CD4+ and CD3+CD4- T cells producing IFNy after immunization with LLOT alone or in the presence of cholera toxin as adjuvant. Cells were analyzed by flow cytometry based on their IFNγ⁺CD3⁺CD4⁺ and IFNγ⁺CD3⁺CD4⁻ expression profile to specify the helper and cytotoxic T-cell populations as IFNy secreting cells. Data were expressed as mean percentage positive cells ± standard deviation. Statistical differences were determined by one-way ANOVA and significant differences were considered at

$$\left(\text{* p} ⩽ 0.05 , \# \text{p} ⩽ 0.005\right)$$

.

FIG. 11. LLO-neutralizing antibodies inhibit L. monocytogenes internalization into hepatocytes. HepG2 cells were incubated for 30 min with wild type (wt) or LLO-deficient L. monocytogenes (Δhly) at MOI = 5, in the presence or absence of increasing concentrations of LLO 528 neutralizing antibodies. L. monocytogenes internalization was measured by fluorescence microscopy. 529 Results are the normalized mean ± SEM of three independent experiments. P values were calculated using 530 a two-tailed Student’s t-test (** = P <0.01).

FIGS. 12A-12B. T cells are required for LLO^{T}+CT immunizations to effectively reduce bacterial burden in mice. Mice were immunized at weekly intervals, for 3 consecutive weeks, by intraperitoneal injection of PBS (negative control), or LLO^{T} (20 µg) plus cholera toxin (1 µg) (LLO^{T} + CT). Mice were given 300 µg of both CD4 and CD8 depleting antibodies or isotype control antibodies on day 26 to deplete T cells via intraperitoneal injection. At day 28, mice were intravenously inoculated with 2 x 10⁴ L. monocytogenes. Mice were given a second 100 µg dose of depleting antibodies or isotype control antibodies 24 h post-infection. Mice were then sacrificed after 72 h to collect blood and enumerate bacterial colony forming units (CFUs) were enumerated in the liver (FIG. 12A) and spleen (FIG. 12B). Data are from 1 experiment with 4 male mice and 2 female mice per experimental condition (indicated in legend). Results are expressed as CFUs/organ and medians are presented. Statistical significance was calculated using a two-sided Mann-Whitney test, N.S. = Not statistically significant, * P < 0.05 ** P < 0.01.

### DETAILED DESCRIPTION

The L. monocytogenes pore-forming exotoxin listeriolysin O (LLO) is an essential virulence factor required for host cell invasion and pathogenesis, with LLO-deficient L. monocytogenes strains being avirulent. Indeed, LLO plays essential roles in the intracellular lifecycle of L. monocytogenes including mediating the disruption of the phagosome to release the bacterium into the host cell cytosol, and mediating the spreading of the bacterium from cell to cell, among other functions.

In addition to its role as a virulence factor, LLO is a major source of CD4+ and CD8+ T cell antigens during the adaptive immune response to L. monocytogenes in mice. CD4+ and CD8+ T cell responses are critical for sterilizing immunity against Listeria monocytogenes. In addition, the passive transfer of LLO neutralizing antibodies can protect naive mice against lethal doses of Listeria monocytogenes. Therefore, disclosed herein is a LLO toxoid-based vaccine that elicits both i) T cell (CD4+ and CD8+) immunity involving LLO antigenic peptides and ii) LLO-neutralizing antibodies, which can efficiently protect humans against Listeria monocytogenes infection. Beyond the interest of developing a listeriosis vaccine, L. monocytogenes and its virulence factor LLO display immune stimulatory functions that have raised considerable interest in the field of cancer immunotherapy. Hence, live-attenuated L. monocytogenes strains have shown promise in providing protection against L. monocytogenes infection and cancer in experimental animal models and several cancer vaccines are currently being tested in clinical trials.

However, the potential dangers of L. monocytogenes live-attenuated strains in immunocompromised individuals have been reported. Given that populations at higher risk for listeriosis and cancer patients are characterized by a weak or altered immunity, a subunit vaccine can prevent the risk of vaccine-related infections. As such, subunit vaccines that utilize important L. monocytogenes virulence factors have been developed. Most of these vaccines induce potent T cell responses (CD4⁺ and CD8⁺), which are essential for the acquisition of sterilizing immunity against L. monocytogenes and play critical roles in anti-cancer immunity.

The pore-forming exotoxin listeriolysin O (LLO) secreted by L. monocytogenes is required for host cell invasion and pathogenesis, with LLO-deficient L. monocytogenes strains being avirulent. Indeed, LLO plays an essential role in the intracellular lifecycle of L. monocytogenes by promoting phagosomal escape of the bacterium into the host cell cytosol. In addition to its role as a virulence factor, LLO has been shown to constitute a major source of CD4⁺ and CD8⁺ T cell antigens during the adaptive immune response to L. monocytogenes in mice. Finally, native as well as non-hemolytic LLO and truncated LLO variants have been shown to stimulate cancer antigen-specific T cell responses. In the present disclosure, a novel LLO toxoid (LLO^{T}) is generated by substituting with Glycine residues a Threonine-Leucine pair located in domain 4, which is critically involved in LLO pore formation. The potency of LLO^{T} as a vaccine antigen alone or in combination with various adjuvants in inducing specific B and T cell responses to protect against L. monocytogenes infection is tested using the murine model.

Described herein is a polypeptide comprising a non-toxic listeriolysin O toxoid that comprises one or more amino acid substitutions at positions 515 and/or 516 relative to SEQ ID NO: 1, and the methods for preventing and treating a Listeria infection.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1 %.

A “composition” is intended to include a combination of active agent and another compound or composition, inert (for example, a detectable agent or label) or active, such as an adjuvant.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington’s Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g. a composition comprising an agent) refers to an amount that is effective to achieve a desired therapeutic result. In some embodiments, a desired therapeutic result is the treatment of a Listeria infection. In some embodiments, a therapeutic result is the prevention of a Listeria infection. In some embodiments, a desired therapeutic result is the treatment of an inflammatory disorder. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “cell membrane”, “plasma membrane”, or “cytoplasmic membrane” as used herein refers to a biological membrane that separates the interior of all cells from the extracellular environment which protects the cell from its environment. Cell membrane is consisted of a lipid bilayer, including cholesterol that sit between phospholipids to maintain their fluidity under various temperature, in combination with proteins. Cholesterol in a plasma membrane may be accumulated in microdomains with specific phospholipids such as sphingomyelin. These domains, often called lipid rafts, ubiquitously distribute from yeast to mammals, playing important roles in cellular functions, such as signal transduction and membrane traffic. In some embodiments, the listeriolysin O toxoid (LLO^{T}) disclosed herein still binds to the host cell membrane despite the destruction of the cholesterol-recognition domain.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed 20 of deoxyribonucleotides.

The term “oligonucleotide” denotes single- or double-stranded nucleotide multimers. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In some embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In some embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

The term “point mutation” means a change in the nucleotide sequence of a gene that results in a single amino acid change in a protein encoded by the gene. For example, a point mutation in a gene can result in the deletion of a single amino acid in a protein encoded by the gene or can result in the substitution of an amino acid in a wildtype version of the encoded protein with a different amino acid. Non-limiting examples of point mutations in listeriolysin O toxoid genes are described herein.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Polypeptides and Polynucleotides

In some aspects, disclosed herein is a polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

It is understood herein that “listeriolysin O” is a member of the largest family of bacterial pore-forming toxins, the cholesterol-dependent cytolysins (CDCs), a hallmark of which is the formation of large oligomeric pores in cholesterol-rich membranes of nucleated cells and erythrocytes. CDC binding to cholesterol is indispensable for the prepore-to-pore transition of the toxin and the cholesterol-binding domain is identified as a conserved Threonine-Leucine pair located in their C-terminal domain 4 (D4). Accordingly, “listeriolysin O toxoid” or the abbreviation “LLO^{T}”, or “non-toxic listeriolysin O” refers to the listeriolysin O toxoid that lacks the conserved Threonine-Leucine motif and displays drastically reduced toxicity. In some embodiments, the listeriolysin O toxoid polypeptide comprises the sequence set forth in SEQ ID NO: 1, or sequence having at or greater than about 80%, about 85%, about 90%, about 95%, about 98%, or about 99% identity with SEQ ID NO: 1, or a polypeptide comprising a portion of SEQ ID NO: 1.

In some embodiments, the amino acid substitution is at amino acid position 515. In some embodiments, the amino acid substitution is at amino acid position 516. In some embodiments, the non-toxic listeriolysin O comprises amino acid substitutions at amino acid positions 515 and 516.

In some embodiments, the amino acid substitutions at amino acid positions 515 and 516 can be, for example, T515G, T515A, L516A, L516G, or any other amino acid substitution(s) that causes the destruction of the cholesterol recognition motif, but does not abolish LLO binding to host membranes. In some embodiments, the amino acid substitution at amino acid position 515 is selected from the group consisting of T515G and T515A. In some embodiments, the amino acid substitution at amino acid position 515 is preferably T515G. In some embodiments, the amino acid substitution at amino acid position 516 can be, for example,T515G, T515A, L516A, L516G, or any other amino acid substitutions that cause the destruction of the cholesterol recognition motif, but do not abolish LLO binding to host membranes. In some embodiments, the amino acid substitution at amino acid position 516 is selected from the group consisting of L516G and L516A. In some embodiments, the amino acid substitution at amino acid position 516 is preferably T516G. In some embodiments, additional amino acid substitutions in listeriolysin O can be used that affect its ability to form pores and/or to bind host cells.

In some embodiments, the polypeptide is isolated. In some embodiments, the polypeptide is recombinant. In some embodiments, the polypeptide is a non-naturally occurring polypeptide.

In some embodiments, the non-toxic listeriolysin O binds to a cell membrane.

In some embodiments, the non-toxic listeriolysin O binds to an antigen-presenting cell.

In some aspects, disclosed herein is a nucleic acid comprising a genetically modified listeriolysin O toxoid gene comprising one or more point mutations, wherein the genetically modified listeriolysin O toxoid gene encodes a polypeptide of any preceding aspect.

In some embodiments, the nucleic acid is isolated. In some embodiments, the nucleic acid is recombinant. In some embodiments, the nucleic acid is a non-naturally occurring nucleic acid.

In some aspects, disclosed herein is a recombinant DNA vector comprising the nucleic acid of any preceding aspect.

In some aspects, disclosed herein is a vaccine comprising: a non-toxic listeriolysin O comprising an amino acid substitution at one or more amino acid positions when compared to SEQ ID NO: 1, wherein the one or more amino acid positions are selected from the group consisting of 515 and 516.

It should be understood that the one or more adjuvants described herein can be any of the adjuvants that can stimulate the production of LLO neutralizing antibodies and T cell immunity. In some embodiments, the vaccine further comprises one or more adjuvants. In some embodiments, the one or more adjuvants are selected from the group consisting of cholera toxin including the β subunit of cholera toxin (CTB), and other detoxified derivatives of cholera toxin. Additional adjuvants can include Freund’s incomplete adjuvant, Freund’s Complete adjuvant, monophosphoryl lipid A, QS-21, salts, i.e., AlK(SO4)2, AlNa(SO4)2, AlNH4(SO4)2, silica, kaolin, carbon polynucleotides, i.e., poly IC and poly AU. Still other adjuvants can include QuilA, Alhydrogel, and the like. Optionally, the vaccine contemplated herein can be combined with immunomodulators that stimulate Toll-like receptors (such as poly(I:C) and CpG motifs) and cytosolic immune sensor (such as cyclic di-nucleotides such as c-di-AMP, c-di-GMP and the like; bacterial mRNA) and immunostimulants (such as interleukins, interferons and the like). Still other adjuvants can include bacterial toxin derivatives. Many vaccine formulations are also known to those of skill in the art.

In some embodiments, the vaccine further comprises a pharmaceutically acceptable carrier.

### Methods of Treatment and Prevention

In some aspects, disclosed herein is a method of preventing, inhibiting, reducing, and/or treating a Listeria infection, comprising administering to a subject an effective amount of a vaccine comprising: a polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

In some aspects, disclosed herein is a method of inducing immune response specific to a Listeria, comprising administering to a subject an effective amount of a vaccine comprising: a polypeptide comprising: a non-toxic listeriolysin O comprising an amino acid substitution at one or more amino acid positions when compared to SEQ ID NO: 1 selected from the group consisting of 515 and 516. It is understood herein that the induced immune response prevents, inhibits, reduces, or treat the Listeria infection.

As the timing of an infection can often not be predicted, it should be understood the disclosed methods of treating, preventing, reducing, and/or inhibiting a Listeria infection, can be used prior to or following the infection of the Listeria infection, to treat, prevent, inhibit, and/or reduce the infection or an infection-associated disease. Where, the disclosed methods can be performed any time prior to the infection. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years, 12, 11, 10, 9, 8, 7, 6, 5, 4, 3, 2, 1 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to infection; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days, 4, 5, 6, 7, 8, 9, 10, 11, 12 or more months, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after infection.

The vaccines of the present invention can be administered to the appropriate subject in any manner known in the art, e.g., orally intramuscularly, intravenously, sublingual mucosal, intraarterially, intrathecally, intradermally, intraperitoneally, intranasally, intrapulmonarily, intraocularly, intravaginally, intrarectally or subcutaneously. They can be introduced into the gastrointestinal tract or the respiratory tract, e.g., by inhalation of a solution or powder containing the conjugates. In some embodiments, the compositions can be administered via absorption via a skin patch. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained.

A pharmaceutical composition (e.g., a vaccine) is administered in an amount sufficient to elicit production of antibodies and activation of CD4+ T cells and CD8+ T cells as part of an immunogenic response. It is understood herein that CD4+ T cell is a group of heterologous lymphocytes having different subsets, including, for example, Th1, Th2, Th17, Tfh, and Treg. In some embodiments, the CD4+ T cell is a Th1 cell. It should also be understood herein that the term “activation” or “activates” refer to a response of a CD4+ T cell, a CD8+ T cell, or a B cell. Such response includes, for example, enhanced proliferation and increased IFN-y+ production of the CD4+ T cell (e.g. Th1), enhanced proliferation and increased IFN-y+ production of the CD8+ T cell, and/or antibody production of the B cell. In some embodiments, administering the vaccine of any preceding aspects activates a CD4+ Th1, a CD8 T cell, and/or a B cell. Dosage for any given patient depends upon many factors, including the patient’s size, general health, sex, body surface area, age, the particular compound to be administered, time and route of administration, and other drugs being administered concurrently. Determination of optimal dosage is well within the abilities of a pharmacologist of ordinary skill.

In some embodiments, the subject is a human. In some embodiments, the human has or is suspected of having Listeria infection. It should be understood herein that the “ Listeria” refers to a genus of bacteria that comprises, for example, L. aquatica, L. booriae, L. cornellensis, L. costaricensis, L. goaensis, L. fleischmannii, L. floridensis, L. grandensis, L. grayi, L. innocua, L. ivanovii, L. marthii, L. monocytogenes, L. newyorkensis, L. riparia, L. rocourtiae, L. seeligeri, L. thailandensis, L. weihenstephanensis, and L. welshimeri. In some embodiments, the Listeria is L. monocytogenes. In some embodiments, disclosed herein is a method of preventing, inhibiting, reducing, and/or treating L. monocytogenes infection.

The vaccine compositions are administered to subjects which may become infected by a Listeria described herein, including but not limited to dogs, cats, rabbits, rodents, horses, livestock (e.g., cattle, sheep, goats, and pigs), zoo animals, ungulates, primates, and humans. In some embodiments, the preferred subject is a human.

### Examples

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Generation of a Full-Length Non-Hemolytic Listeriolysin O Toxoid (LLO^{T})

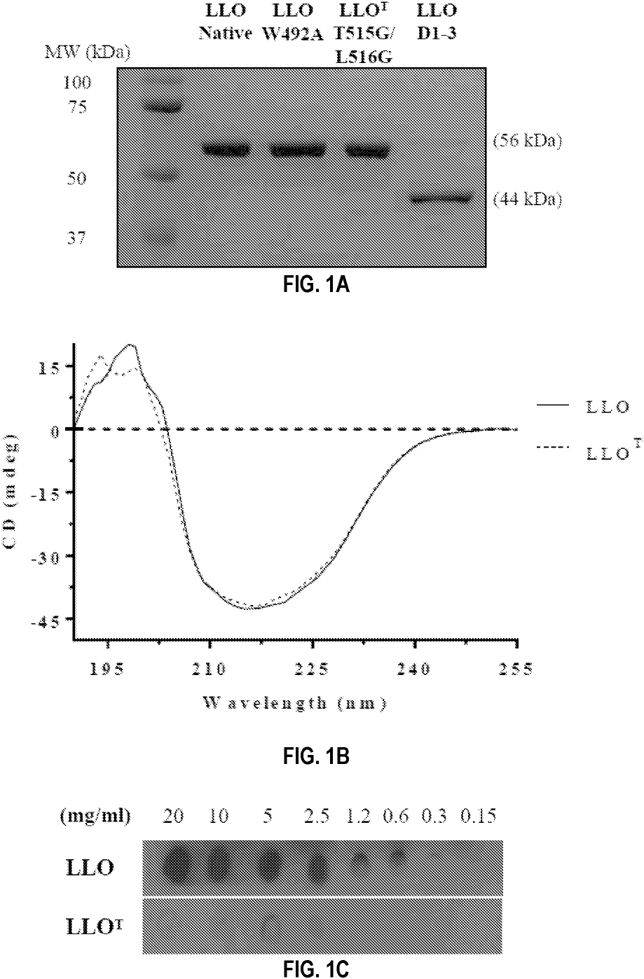

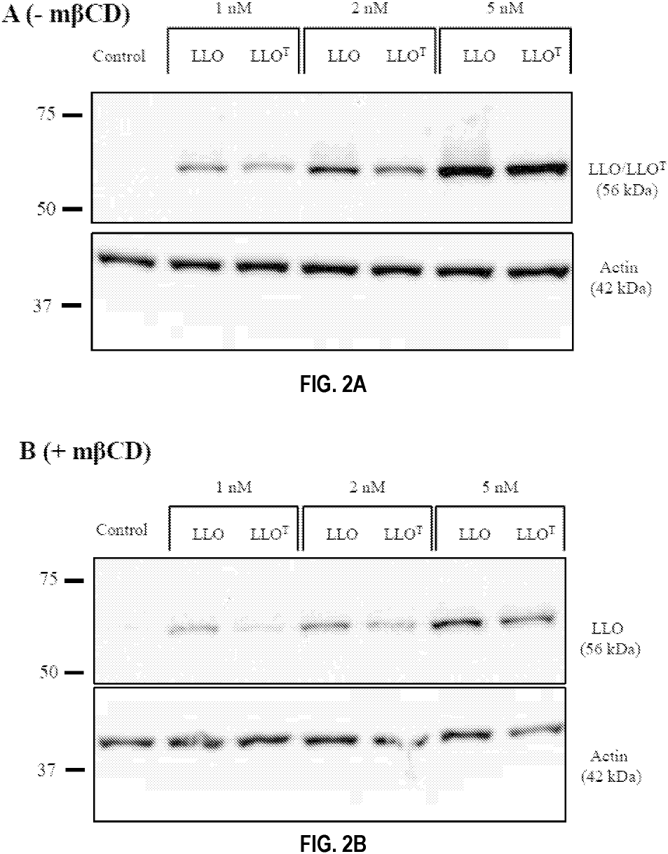

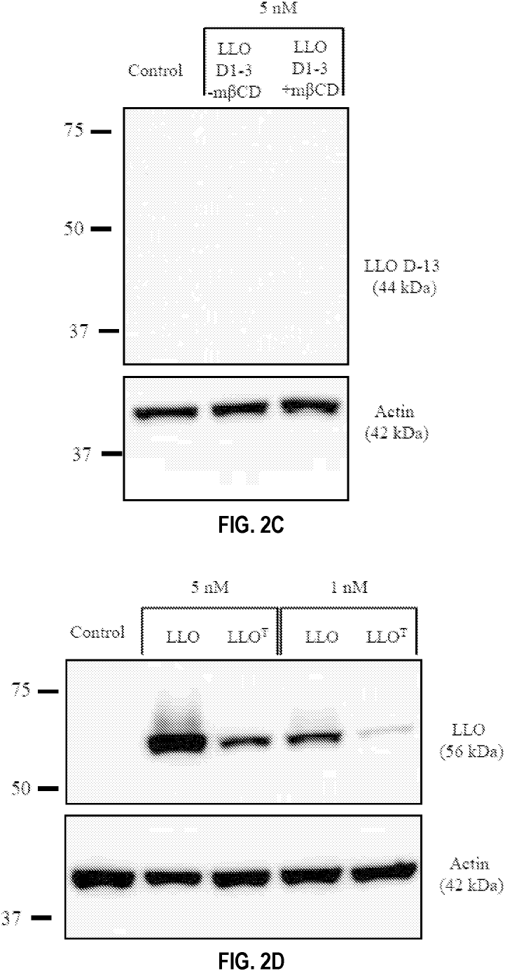

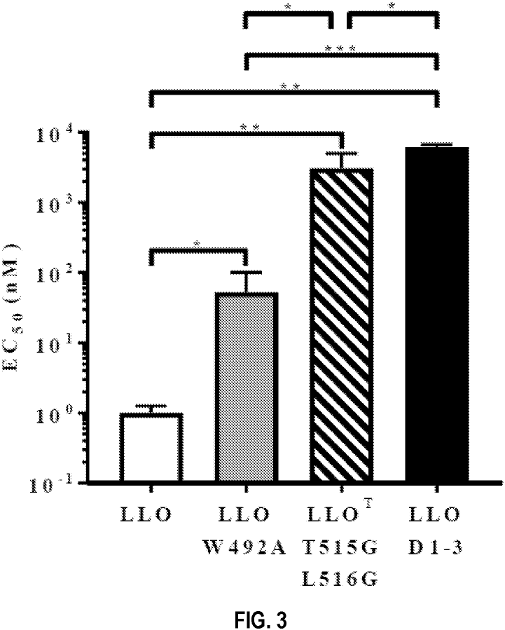

LLO is a member of the largest family of bacterial pore-forming toxins, the cholesterol-dependent cytolysins (CDCs), a hallmark of which is the formation of large oligomeric pores in cholesterol-rich membranes of nucleated cells and erythrocytes. CDC binding to cholesterol is indispensable for the prepore-to-pore transition of the toxin and the cholesterol-recognition domain was identified as a conserved Threonine-Leucine pair located in their C-terminal domain 4 (D4). A full length LLO toxoid (LLO^{T}) was generated by substitution of the cholesterol-recognition threonine-leucine pair with glycines (T515G/L516G). The properties of LLO^{T} was compared relative to native LLO, a truncated LLO D1-3 variant devoid of the host cell binding domain D4, and a full-length LLO variant with the amino acid substitution W492A in domain 4 that was previously reported as non-hemolytic (LLO W492A). Recombinant 6-histidine-LLO, -LLO^{T}, -LLO D1-3, and -LLO W492A were purified and characterized by SDS-PAGE (FIG. 1A). Circular dichroism compared LLO^{T} to LLO (FIG. 1B). The spectra for LLO^{T} and LLO were similar, indicating that the toxoid is properly folded (FIG. 1B). A dot blot assay confirmed that while LLO bound cholesterol, LLO^{T} was unable to bind cholesterol (FIG. 1C). Binding of LLO^{T} to host cell membranes was then tested, since most CDCs such as PFO, PLY, and SLO are unable to bind human erythrocytes in the absence of the cholesterol-recognition motif. LLO^{T}, however, retained binding to HeLa (human epithelial cell line) and THP-1 (human monocyte cell line) cells, though not to the same extent as native LLO. Indeed, 2 nM LLO^{T} provided equivalent binding to HeLa cells as 1 nM LLO while 1 nM LLO and 5 nM LLO^{T} provided equivalent binding to THP-1 cells (FIGS. 2A and 2D). Importantly, LLO D1-3 did not bind to host cells, confirming that host cell binding was only mediated by domain 4 (FIG. 2C). When cholesterol was depleted by treatment with mβCD, host cell binding of both LLO and LLO^{T} was reduced, but not abrogated (FIG. 2B). Together, these results establish that cholesterol is a host cell ligand for LLO, but suggest the presence of additional ligands bound by LLO D4. Furthermore, cholesterol indirectly affects LLO binding to host cells, for example by affecting the biophysical properties of the plasma membrane and/or access of LLO to other membrane ligands. Finally, hemolytic activity of LLO^{T} was markedly decreased compared with native LLO and LLO W492A. There was an approximate 3,000-fold and 60-fold, decrease in hemolytic activity of LLO^{T} compared to native LLO and LLO W492A, respectively. These results show the indispensable role of cholesterol and the threonine515-leucine 516 pair in LLO pore formation (FIG. 3). LLO^{T} hemolytic activity was nearly as low as the truncated LLO D1-3 variant, which is totally unable to bind host cells (FIG. 3). These results confirm the indispensable roles of cholesterol and the threonine-leucine pair for LLO pore formation. In conclusion, LLO^{T} retains its antigenic peptides, proper folding, and ability to bind to host cells (including antigen-presenting cells), which are critical features for efficient capture and presentation by antigen presenting cells.

### Example 2. Immunization with LLO^{T} and Cholera Toxin Protects Mice against L. Monocytogenes

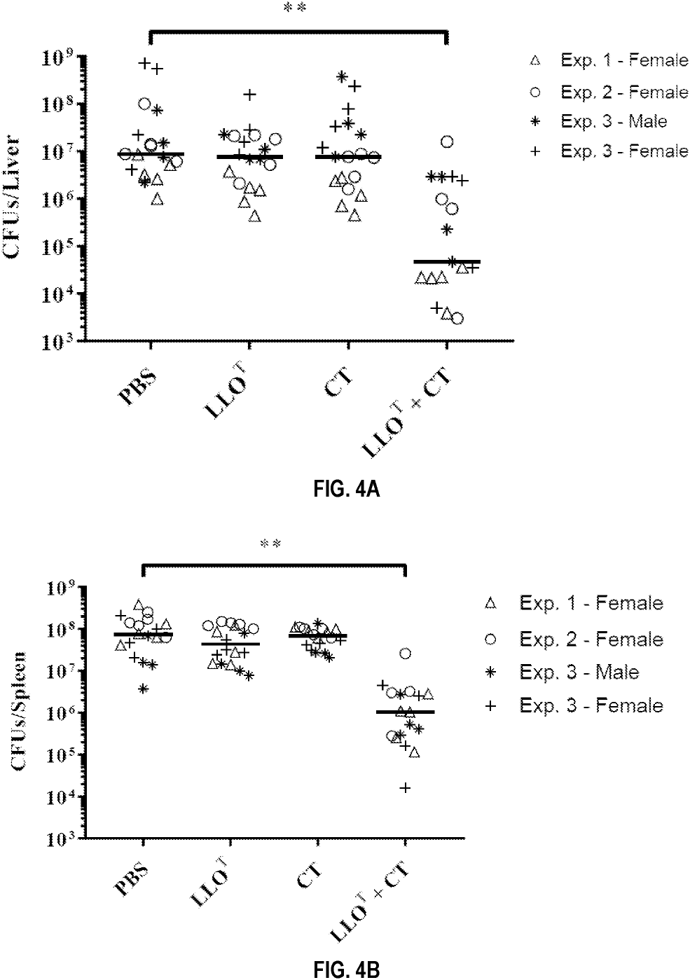

Sterilizing immunity against L. monocytogenes is well known to involve CD4⁺ and CD8⁺ T cells. In addition, the passive transfer of monoclonal LLO-neutralizing antibodies was shown to efficiently protect naive mice against sub-lethal and lethal doses of L. monocytogenes. To determine if LLO^{T} can promote immunization of mice against L. monocytogenes, LLO^{T} was administered in the presence or absence of the experimental adjuvant cholera toxin (CT). Cholera toxin was selected for its broad effects on stimulating both T and B cell responses. Mice were treated with PBS, LLO^{T}, CT, or LLO^{T} + CT via intraperitoneal injections at weekly intervals for 3 weeks. At day 28 after initial immunization, mice were challenged with 2 x 10⁴ L. monocytogenes by tail vein injection and bacterial burden was determined by CFU enumeration in the spleen and liver three days post-infection. As shown in FIGS. 4A-4B, mice immunized with LLO^{T} + CT were significantly protected against L. monocytogenes when compared to the groups that received LLO^{T} alone, CT alone, or PBS.

### Example 3. Immunization with LLO^{T} and Alum does not Protect Mice against L. Monocytogenes

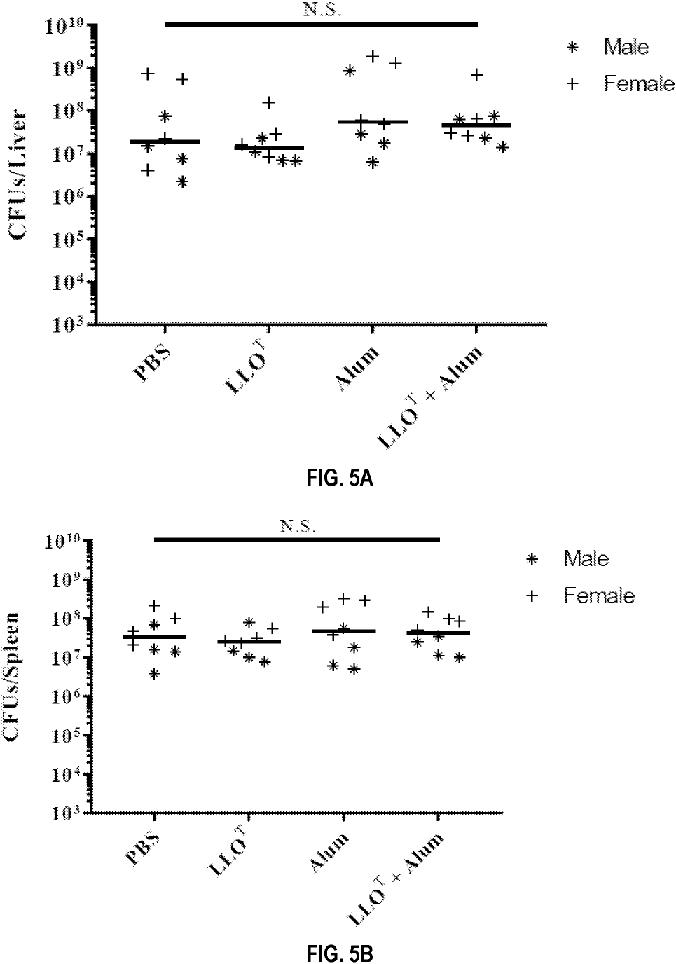

To test whether production of anti-LLO antibodies alone can play a role in the protection of mice, the effectiveness of Alum was examined. Alum is a widely used vaccine adjuvant that predominantly induces strong Th2 responses and antibody responses to antigens. After a similar immunization procedure as described previously with CT, mice that received LLO^{T}+ Alum were not protected against L. monocytogenes as previously observed with CT + LLO^{T} (FIGS. 5A-5B).

### Example 4. Immunization With LLO^{T} and Cholera Toxin, but not Alum, Leads to the Production of LLO-Neutralizing Antibodies

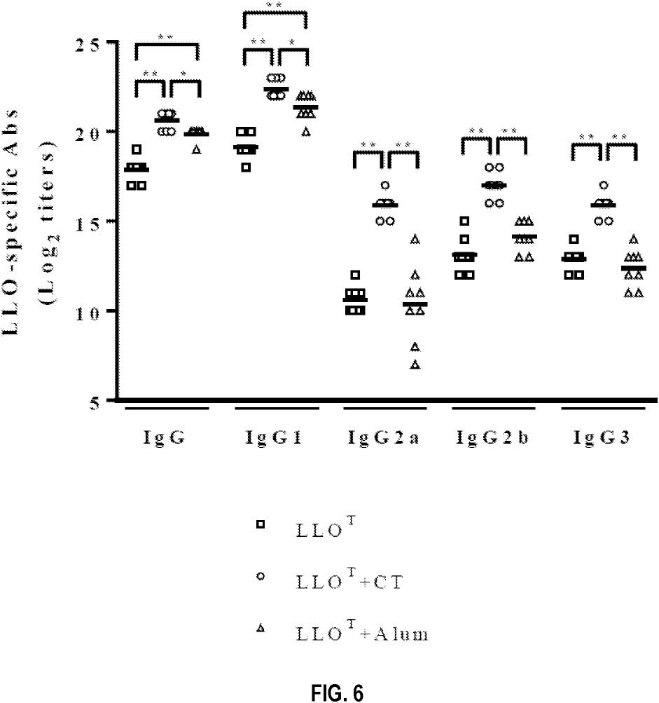

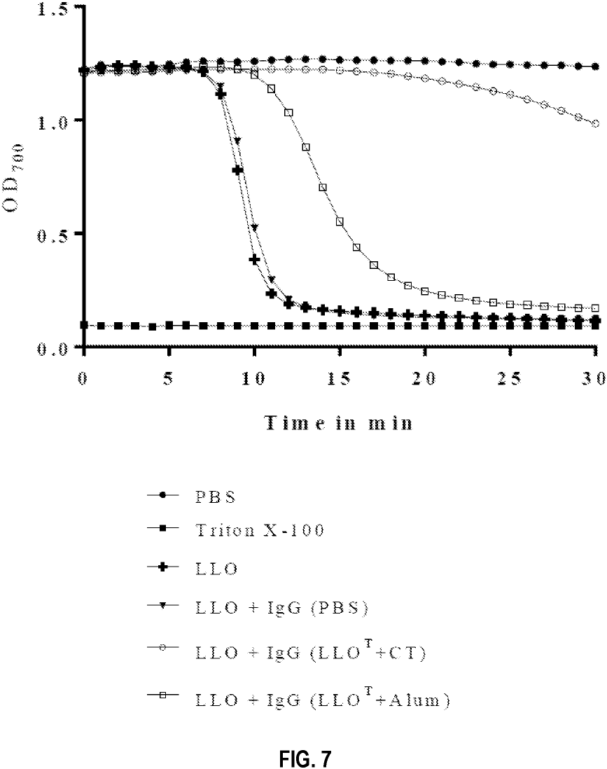

To address the difference in the ability of CT and Alum as adjuvants to protect mice immunized with LLO^{T} against L. monocytogenes challenge, the production of anti-LLO^{T} IgG titers in the various groups of animals was measured. Data summarized in FIG. 6 showed that mice immunized with LLO^{T} alone or with LLO^{T} + CT produced anti-LLO^{T} IgG. However, the adjuvant greatly enhanced the production of LLO^{T}-specific IgG including IgG1 and IgG2a. Furthermore, mice immunized with LLO^{T} + CT produced more IgG1 than IgG2a, a profile previously seen when CT was administered with inert antigens such as ovalbumin. Mice immunized with LLO^{T} + Alum developed lower levels of LLO^{T}-specific IgG1 responses than those given LLO^{T}+ CT (FIG. 6). In contrast to CT, Alum did not enhance the levels of IgG2a, IgG2b, or IgG3 compared to LLO^{T} administered alone (FIG. 6). Indeed, LLO^{T} alone or LLO^{T} + Alum led to similar levels of anti-LLO IgG2a, IgG2b, and IgG3. Finally, the IgGs purified from mice treated with LLO^{T} + CT efficiently neutralize LLO activity, which was not observed with IgGs purified from mice treated with LLO^{T} + Alum (FIG. 7). Together, these data show that unlike Alum, CT induced efficient production of anti-LLO IgG2a/c, IgG2b and IgG3 isotypes and neutralizing anti-LLO antibodies.

### Example 5. The Protective Immunization with Cholera Toxin, but not with Alum, Elicits A Pronounced Increase in Th1 Type Responses to LLO

To establish the nature of the T helper responses elicited by the protective (LLO^{T} + CT) and non-protective (LLO^{T} + Alum) vaccine formulations, in comparison to LLO^{T} alone and control PBS, splenocytes were collected from the different groups of mice and in vitro stimulated with LLO^{T}. After 5 days of culture, cells were extracellularly stained with fluorescent anti-CD3 and anti-CD4 antibodies to denote CD4⁺ T helper cells, intracellular stained with fluorescent antibodies to identify Th1 (IFN-γ, and TNF-α)-, Th2 (IL-5, IL-4, and IL-10)-, Th17 (IL-17A)-, and Tfh (IL-21)-type cytokines. This labeling strategy can also characterize the production of cytokines by CD8+ T cells, identified as CD3⁺CD4⁻ cells that were positive for any of the tested cytokines.

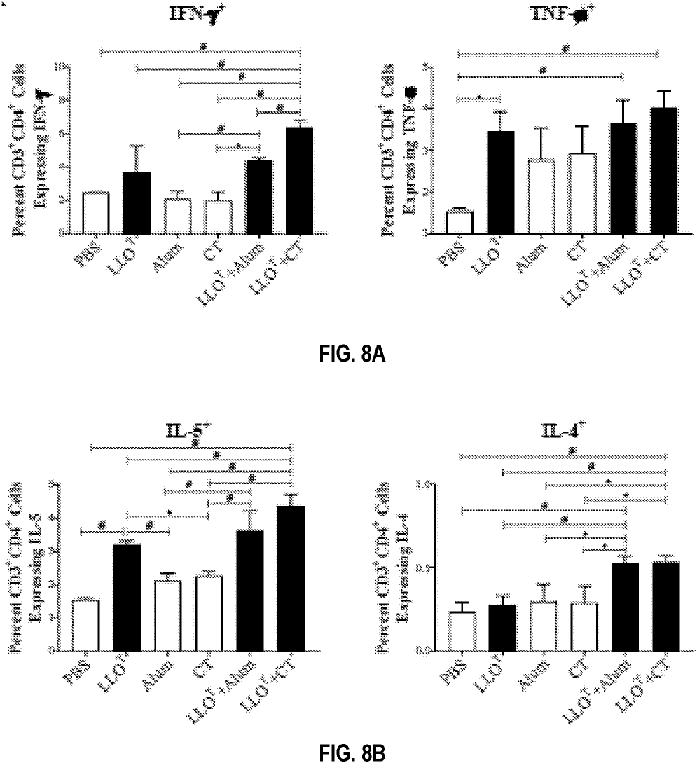

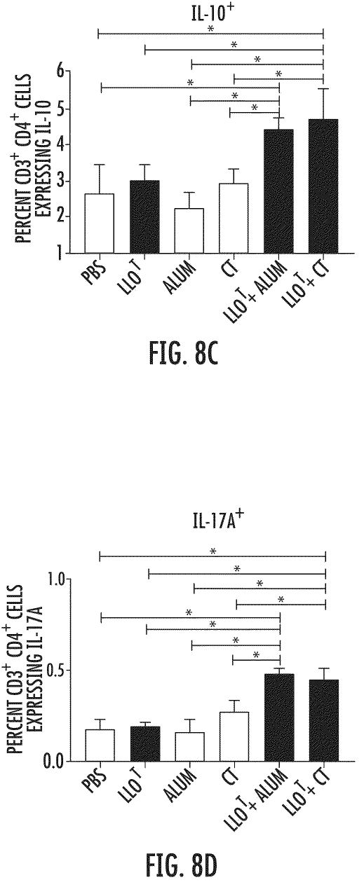

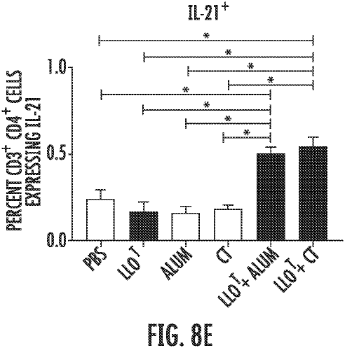

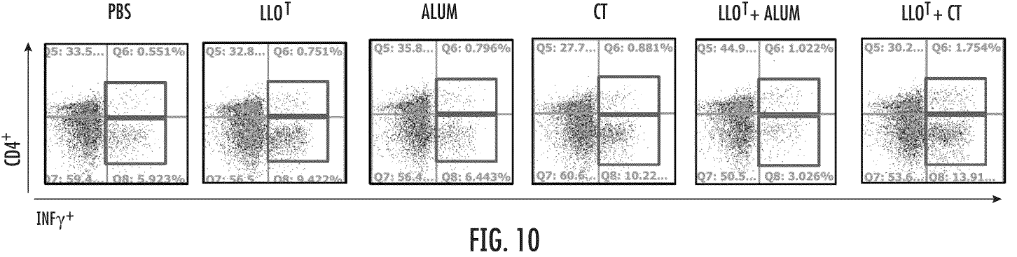

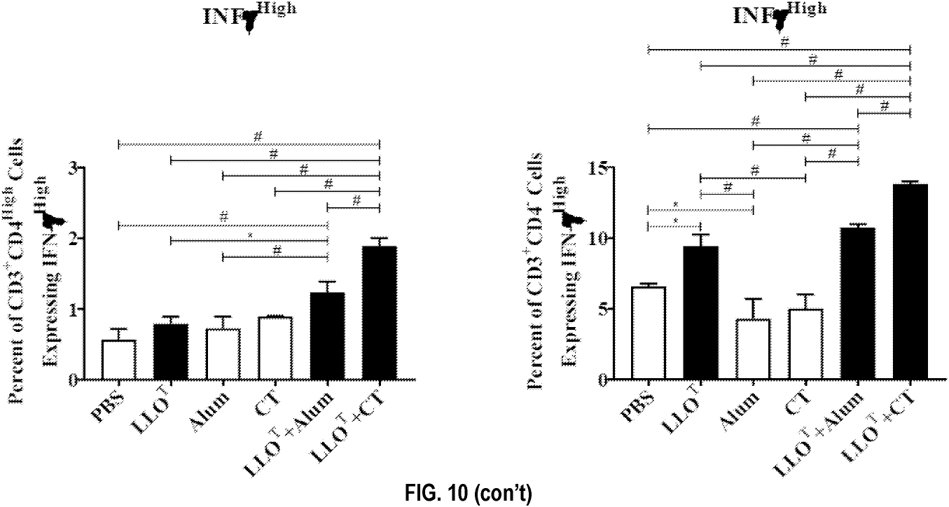

Th1 cells and their characteristic cytokines (IFN-γ and TNF-α) promote cell-mediated immunity, including cytotoxic CD8⁺ T cells and the activation of macrophages, both of which are important for protection against intracellular pathogens, including L. monocytogenes. Flow cytometry analysis of CD4⁺ CD3⁺cells (CD4⁺ T cells) showed that immunization with LLO^{T} + CT led to a significant increase in IFN-y producing T helper cells when compared to all other treatments (FIG. 8A). Immunization with LLO^{T} + Alum led to a significant increase in IFN-y producing T helper cells over Alum, or CT control groups; however, this increase was still significantly lower when compared to LLO^{T} + CT (FIG. 8A). Immunization with LLO^{T} in the presence or absence of adjuvants led to a significant increase in TNF-α producing T helper cells when compared to the PBS negative control (FIG. 8A). Also, LLO^{T} along can elicit significant increase in IFN-y production by CD3⁺CD4⁻ CD8⁺ T cells (FIG. 10).

Th2 cells are known to produce cytokines (IL-5, IL-4, and IL-10) that support the production of antibodies. The main products of Tfh cells (IL-21) and Th17 cells (IL-17A) also facilitate antibody production and their affinity maturation. Immunization with LLO^{T} + Alum or CT led to significant increases in cytokine producing T helper cells when compared to the PBS control group and the groups given the adjuvants alone. Additionally, immunization with LLO^{T} alone led to increases in IL-5 producing T helper cells (FIGS. 8B-8E). Taken together, the results indicate that both the non-protective Alum and the protective CT adjuvants lead to increased Th1, Th2, and Th17 responses. However, the protective immunization with LLO + CT leads to the most pronounced increase in IFN-y responses when compared to all other conditions, including the condition in which the LLO^{T} alone (which is non-protective) was used for immunization.

### Example 6. Immunization with LLO^{T} and Cholera Toxin as Adjuvant Protects Mice Lacking Mature B Cells against L. Monocytogenes

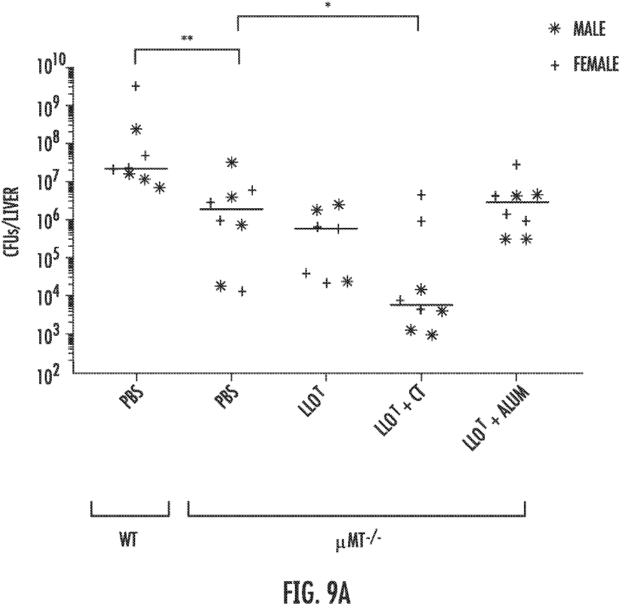

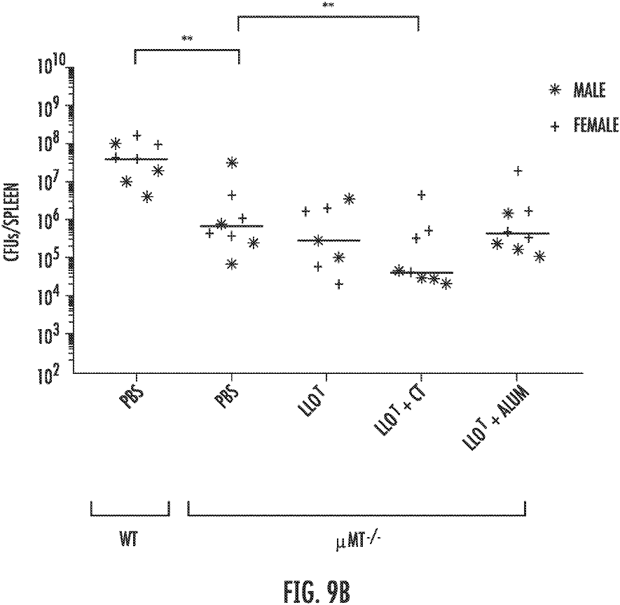

The protective immunization regimen (LLO^{T} + CT) was characterized by both increased LLO-specific neutralizing antibody production (FIG. 7) and Th1 responses (FIGS. 8A-8E) when compared to the non-protective (LLO^{T}+ Alum) treatment. To establish if the production of anti-LLO antibodies had a significant role in protection against L. monocytogenes in the immunized group, the immunization procedure was repeated using mice that lack mature B cells µMT^{-/-}) in comparison to wild type mice. Regardless of treatment, there was a significant reduction in bacterial burden in µMT^{-/-} mice compared to WT mice, as previously reported in the literature. LLO-specific IgGs in µMT^{-/-} mice were not detected; whereas LLO-specific IgGs were being induced in WT mice by LLO^{T} + CT as previously observed. Despite the lack of LLO-specific antibody production in µMT^{-/-} mice, LLO^{T} + CT still induced significant protection against L. monocytogenes (FIGS. 9A-9B). Therefore, LLO-neutralizing antibodies are dispensable for protection against L. monocytogenes, which does not exclude that when present the LLO neutralizing antibodies reinforce the antibacterial action of the immune response.

### Example 7. Materials and Methods

Generation of LLO variants and LLO toxoid. The gene coding for six-His-tagged LLO^{T} with the substitutions T515G and L516G was generated by PCR-based site-directed mutagenesis using the pET29b plasmid harboring wild type hly (the gene coding LLO) as a template and mutagenic primers (Forward - 5-gaa ata tct cca tct ggg gca ccg ggg gtt atc cga aat ata gta ata aag-3 (SEQ ID NO:2) and Reverse - 5-ctt tat tac tat att tcg gat aac ccc cgg tgc ccc aga tgg aga tat ttc-3 (SEQ ID NO:3)) as described previously. The gene coding for six-His-tagged LLOW492A was also generated using the same strategy and the mutagenic primers (Forward - 5-ggt tta gct tgg gaa tgg gcg aga acg gta att gat gac cgg-3 (SEQ ID NO:4) and Reverse - 5-ccg gtc atc aat tac cgt tct cgc cca ttc cca agc taa acc-3 (SEQ ID NO:5)). The gene coding for the six-His-tagged truncated listeriolysin O LLO (LLO D1-3) was amplified by PCR from the wild type sequence of hly using the Forward - 5′-aac gtg cat atg gat gca tct gca ttc aat aaa G-3′ (SEQ ID NO:6) and Reverse - 5′-att ctc gag tgt ata agc ttt tga agt tgt-3′ (SEQ ID NO:7) and cloned into pET29b using NdeI and XhoI restriction sites. LLO variants were purified. LLO variants were aliquoted in 50 mM phosphate, pH=6, 1 M NaCl and stored at -80° C. until used. Endotoxin measurements were performed as directed by the manufacturer using the Chromogenic Endotoxin Quant Kit (Pierce), and LLO^{T} was inoculated at 200 µg/ml with endotoxin levels strictly below the recommended limit of 36 EU/ml. For detection of the toxin derivatives by SDS-PAGE, 1 µg of recombinant LLO, LLOW492A, LLO^{T}, or LLOD1-3 were diluted in Laemmli sample buffer with β-mercaptoethanol and denatured by heating at 95° C. for 5 min. Samples were subjected to sodium dodecyl sulfate polyacrylamide gel electrophoresis (SDS-PAGE) in 10% polyacrylamide gels. Gels were stained with Coomassie blue and imaged using a ChemiDoc XRS imaging system (Bio-Rad).

Circular Dichroism (CD) spectroscopy. CD spectra for LLO and LLO^{T} were acquired on a Jasco J-815 spectrometer at 10° C. with a 1 mm cuvette at a protein concentration of 0.5 mg/ml in 20 mM sodium phosphate buffer at pH=6. Spectra were recorded at wavelength intervals of 1 nm (190 to 255 nm). The spectra are the average of 3 scans

Cholesterol Binding Assay. Spots (2 µl) of a serially diluted ethanol-cholesterol solution were deposited onto a PVDF membrane and air-dried. The membranes were saturated by incubation in a 20 mM Tris buffer (TBS) containing 4% nonfat milk and 0.2% Tween 20 at pH 7.4. LLO and LLO^{T} (20 µg/ml) were incubated at 4° for 3 h in TBS with 0.2% Tween 20. After washes, rabbit anti-LLO Abs (Abcam) were incubated for 1 h in TBS with 0.1% Tween 20, followed by washes and incubation with horseradish peroxidase (HRP)-conjugated secondary antibodies in TBS with 0.1% Tween 20. LLO was detected with ECL Western Blotting Detection Kit (Amersham). Western blotting was performed to verify that the rabbit anti-LLO antibodies recognize LLO and LLO^{T} with similar efficiency.

LLO binding assays HepG2 invasion assays. HeLa cells (1.5 x 10⁵ /well) were grown for 24 h in 6-well plates in Dulbecco’s modified Eagle’s medium (DMEM) supplemented with 10% heat-inactivated fetal bovine serum (HIFBS), 100 U/ml penicillin and 100 µg/ml streptomycin (Invitrogen). Cells were incubated for 30 min in FBS-free medium +/- 5 mM methyl-β-cyclodextrin (mβCD) at 37° C. to deplete cholesterol. Cells were incubated for 10 min in FBS-free medium with LLO, LLO^{T}, or LLO D1-3 at 1, 2, or 5 nM at 4° C. Cells were then washed with PBS and lysed with lysis buffer (150 mM NaCl, 20 mM Tris/HCl, 2 mM EDTA, 1% NP-40, and protease inhibitor cocktail (Roche)). Cell lysates were subjected to western blot analysis using an anti-LLO (Rabbit polyclonal from Abcam) or anti-actin antibodies (Cell Signaling) and secondary antibodies conjugated to HRP (Cell Signaling). Detection was performed using the Amersham ECL Select Reagent Kit (GE Healthcare) and a ChemiDoc XRS Imaging System (Bio-Rad). THP-1 cells were cultured in RPMI-1640 supplemented with 10% HIFBS, 100 U/ml penicillin and 100 µg/ml streptomycin (Invitrogen). 2 x 10⁶ cells were washed with FBS-free medium and incubated with 1 nM and 5 nM LLO or LLO^{T} for 10 min at 4° C. THP-1 cells were washed with PBS, lysed and subjected to western blot analysis as described above. For invasion assay, HepG2 cells were cultured in 24-well plates and incubated with bacteria at multiplicity of infection 5 for 30 min in the presence or absence of LLO-neutralizing antibodies. Cells were washed, fixed and labeled to measure the percentage of bacterial internalization as described in 31. All human cell lines used in this study were authenticated by ATCC.

Hemolysis Assays. Human blood was drawn in heparinized Vacutainer tubes, from healthy adult volunteers with approval of the Ohio State University Institutional Review Board. After centrifugation of blood on Polymorphprep (Axis-Shield, Oslo, Norway), erythrocytes were collected from the lower cell layer and were washed with Alsever’s solution. The concentrations of LLO and its derivatives leading to 50% hemolysis (EC₅₀) were determined by performing a hemolysis assay as follows. Erythrocytes were washed three times with phosphate buffered saline (PBS) and diluted to a concentration of 4 x 10⁷ cells/ml. Duplicate serial dilutions of native LLO, LLOW492A, LLOD1-3, and LLO^{T} were made at 4° C. in a round bottom 96-well plate, and 160 µl of cold erythrocytes suspension were added in each well. Concentration ranges tested were: native LLO (100 nM - 0.1 nM), LLOW492A (3,000 nM - 1.5 nM), LLO^{T} (10,000 nM - 5 nM), LLOD1-3 (6,000 nM - 3 nM). Plates were then incubated for 30 min at 37° C., centrifuged, and the supernatants were transferred to a flat bottom 96-well plate for reading their absorbance (540 nm) in a spectrophotometer. Erythrocytes were treated with 0.1% Triton X-100 (100% hemolysis) and with PBS (no hemolysis) as positive and negative controls, respectively. The concentration of toxin leading to 50% hemolysis (EC₅₀) was determined by polynomial regression using Graph Pad Prism 7 software (GraphPad Software Inc, La Jolla, CA).

Immunization. All animal protocols were approved by The Ohio State University’s Institutional Laboratory Animal Care and Use Committee. Seven to eight week-old C57BL/6 or C57BL/6-Igh-6^{tm1Cgn} (B cell-deficient, also known as µMT^{-/-})³⁰ mice, purchased from The Jackson Laboratory (Bar Harbor, ME), were housed in the university vivarium for one week before starting immunization. Mice were immunized on days 0, 7, and 14 by intraperitoneal injection of 100 µl of injectable grade PBS containing one of the following: 20 µg LLO^{T} alone, 20 µg LLO^{T} plus 1 µg cholera toxin (List Biological Laboratories, Inc, Campbell, CA), 20 µg LLO^{T} adsorbed on 40 µg alum (ThermoFisher Scientific, Waltham, MA). Control groups received 100 µl of PBS alone, or 1 µg cholera toxin, or 40 µg of alum. For the preparation of alum plus LLO^{T}, LLO^{T} was adsorbed to alum via gentle mixing for 45 min at 4° C. Blood was collected from mice via submandibular cheek bleed during the immunization procedure on days 14, 21, and 28. Serum was obtained by centrifugation of the clotted blood (1,500 x g for 15 min at 4° C.). For IgG isolation, larger volumes of blood were obtained via cardiac puncture immediately after sacrifice of the animals.

Bacterial Cell Culture and Mouse Infection Wild type L. monocytogenes (strain DP10403S) were grown overnight at 37° C. in brain heart infusion (BHI). For infections, overnight cultures were diluted 1/20 in BHI and grown at 37° C. until OD₆₀₀ = 0.7-0.8. Bacteria were washed three times and diluted in injectable grade phosphate-buffered saline (PBS). Mice were inoculated by tail vein injection with L. monocytogenes (2 x 10⁴ bacteria in 100 µl injectable grade PBS) on day 28 after immunization. After 72 h, mice were euthanized and livers, spleens, and blood were collected. Organs were homogenized in PBS and homogenates were serially diluted, plated on BHI agar plates and incubated at 37° C. for 48 hours. Bacterial colonies were enumerated to determine the colony forming units (CFUs).

Evaluation of LLO-specific antibody titers. To determine the LLO-specific antibody titers, ELISA was performed with LLO-coated plates. Briefly, 100 µl of LLO^{T} (5 µg/ml in PBS) were added to microtiter plates and incubated at 4° C. overnight. Plates were washed three times with cold PBS and blocked for 2 h with 1% BSA in PBS. Plates were washed three times and 100 µl of PBS 1% BSA containing serial dilution of sera were added. After overnight incubation at 4° C., the LLO^{T}-specific antibodies were detected with HRP-conjugated anti-mouse IgG sera (1:3000 dilution) (Southern Biotech Associates Inc., Birmingham, AL). Alternatively, to measure IgG subclasses, biotin-conjugated rat anti-mouse IgG1, IgG2a/c, IgG2b, or IgG3 monoclonal Abs and HRP-conjugated streptavidin (BD Biosciences, San Jose, CA) were used (0.5 µg/ml). The HRP substrate ABTS (2,2′-Azinobis [3-ethylbenzothiazoline-6-sulfonic acid]-diammonium salt, Sigma-Aldrich) was added and the antibody titers were determined as the last dilution of samples with an absorbance of >0.1 above that of samples from control mice mock immunized with PBS.

Evaluation of the production of LLO-neutralizing antibodies. To test for LLO neutralization by LLO^{T}-induced antibodies, a kinetic hemolytic assay was performed. IgG were purified from serum collected from immunized mice using protein G-agarose (Pierce) according to the manufacturer’s instructions. LLO and LLO^{T} (5 nM in PBS) and various dilutions of purified serum IgG were pre-incubated on ice in a 96-well plate for 15 min before the addition of erythrocytes at 4 x 10⁷ cells/ml, to test LLO activity using a kinetic assay. Triton X-100 (0.1%) and PBS served as positive and negative controls for hemolysis, respectively. Samples were transferred to a spectrophotometer at 37° C. and the absorbance (700 nm) was measured every minute for 30 min.

Analysis of LLO^{T}-specific T helper cell cytokines responses. Spleens were aseptically removed from mice 38 days after initial immunization and minced by pressing through a cell strainer. Red blood cells were removed by incubation in 0.84 % ammonium chloride and, following a series of washes in RPMI 1640, spleen cells were suspended in RPMI 1640 supplemented with 2 mM L-glutamine, 1 mM sodium pyruvate, 10 mM HEPES, 100 U/ml penicillin, 100 µg/ml streptomycin, and 10% fetal calf serum. The cell concentration was adjusted to 5 × 10⁶ cells/ml, and 100 µl cells were added to each well (3 wells per spleen) of a 96-well micro-titer plate and cultured either alone or in the presence of 5 µg/ml LLO^{T} for 5 days at 37° C. in a 5% CO₂ atmosphere. Flow cytometry and intracellular cytokine staining were then used to determine the profile of T helper cell cytokine responses. For this purpose, cells were stimulated with PMA and Ionomycine (BD-Pharmangen, NJ, US) and incubated for 1 h at 37° C. in a 5% CO₂ atmosphere. The Golgi function was blocked by Golgistop, (BD-Pharmangen, NJ, US), and cells were incubated at 37° C. in a 5% CO₂ atmosphere for 5 h. Cells were then collected and washed twice with FACS buffer (PBS, 2% BSA, 0.01% NaN₃). For labeling extracellular T-cell lineage markers, cells were incubated with Alexa Fluor 700 anti-CD3 and Alexa Fluor 750 anti-CD4 antibodies (Biolegend, San Diego, CA) for 30 min at 4° C., then washed twice with FACS buffer. For intracellular cytokine staining, cells were incubated with Fixation-Permeabilization Buffer (BD-Pharmagen, NJ, US) for 20 min at 4° C. and washed twice with the permeabilization buffer (BD-Pharmangen, NJ, US). Cells were then labeled with Th1, Th2, Th17, and Tfh cytokine-specific antibodies (Alexa Fluor 488-IFNy, PerCP Cy5.5-TNFα, PE-IL-5, Alexa Fluor 647-IL-21, PECy7 IL-10, Brilliant Violet 650 IL-17, Brilliant Violet 605 IL-4 (Biolegend, San Diego, CA)) for 30 min at 4° C. Cells were washed twice with the permeabilization buffer and then washed twice with the FACS buffer. Cells were suspended in FACS buffer and analyzed with an Attune NxT flow cytometer (Thermo Fisher Scientific, Waltham, MA). The data were analyzed by triple gating as (CD3⁺CD4⁺Cytokines⁺). Statistical analyses were performed by one-way ANOVA using Graph Pad Prism7 (GraphPad Software Inc, La Jolla, CA) and significant differences were considered at p ≤ 0.05 (*) .

### Example 8. Full-length LLO Toxoid (LLO^{T}) in which the Thr-Leu (T515G/L516G) Cholesterol Recognition Motif in Domain 4 was Substituted.

Disclosed here in is the generation of a full-length LLO toxoid (LLO^{T}) in which the Thr-Leu (T515G/L516G) cholesterol recognition motif in domain 4 was substituted with two glycine residues. Using LLO^{T} and the cholera toxin experimental adjuvant, a novel vaccine was created that protects against infection by L. monocytogenes. This vaccine elicits CD4⁺ Th1 and CD8⁺ cells producing IFN-y and B cells producing LLO-neutralizing antibodies. The advantages of developing a LLO^{T}-based subunit vaccine are safety, the fact that LLO^{T} binds antigen-presenting cells and contains all native antigens for efficient activation of T and B cell responses, while LLO toxicity is abrogated. Finally, this vaccine elicited a response that neutralizes LLO, which is the most critical virulence factor of the bacterium.

The cholesterol recognition motif is conserved among the cholesterol-dependent cytolysin (CDC) family members and was shown to be essential for perfringolysin O (PFO), streptolysin O (SLO), pneumolysin (PLY), and intermedilysin (ILY) binding to cholesterol. The data herein show that this motif is also required for LLO binding to cholesterol (FIGS. 1A-1C). Most CDCs including PFO, PLY, and SLO bind host cells in a cholesterol-dependent fashion. They are unable to bind cholesterol-depleted cells, or to bind host cells in the absence of the cholesterol recognition motif. However, for a few CDC members including ILY, binding to host cells is cholesterol-independent. Despite the absence of the cholesterol recognition motif or despite cholesterol depletion, LLO^{T} bound to host cell membranes (FIGS. 2A-2D). This indicates the presence of additional unidentified host receptors for LLO. LLO^{T} displayed drastically reduced hemolytic activity. Indeed, LLO^{T} hemolytic activity was as low as a truncated LLO variant lacking the entire membrane-binding domain (FIG. 3). The loss of toxicity, the maintenance of LLO membrane binding and the preserved presence of T cell antigens make LLO^{T} an excellent subunit vaccine against anti-L. monocytogenes. LLO and its non-hemolytic derivatives display immunogenic properties. When used as an adjuvant with a dengue virus antigen, a detoxified LLO variant (carrying mutations in the consensus undecapeptide sequence, which is critical for pore formation) increased production of dengue virus envelope protein-specific IgG1 and IgG2a. This particular LLO variant proved effective as an adjuvant for tumor immunotherapy in mice. The data disclosed herein show that LLO^{T} displays immunogenic properties as it elicits significant production of LLO-specific IgG1, IgG2a/b/c and significant increase in TNF-α and IL-5 producing T cells. Also, LLO^{T} alone is likely able to induce CD8⁺ cytotoxic responses since a proportion of CD4⁻ CD3⁺ cells produced IFN-y in response to LLO^{T}. However, in the present experimental model, LLO^{T} alone was not sufficient to protect mice against L. monocytogenes and required adjuvant.

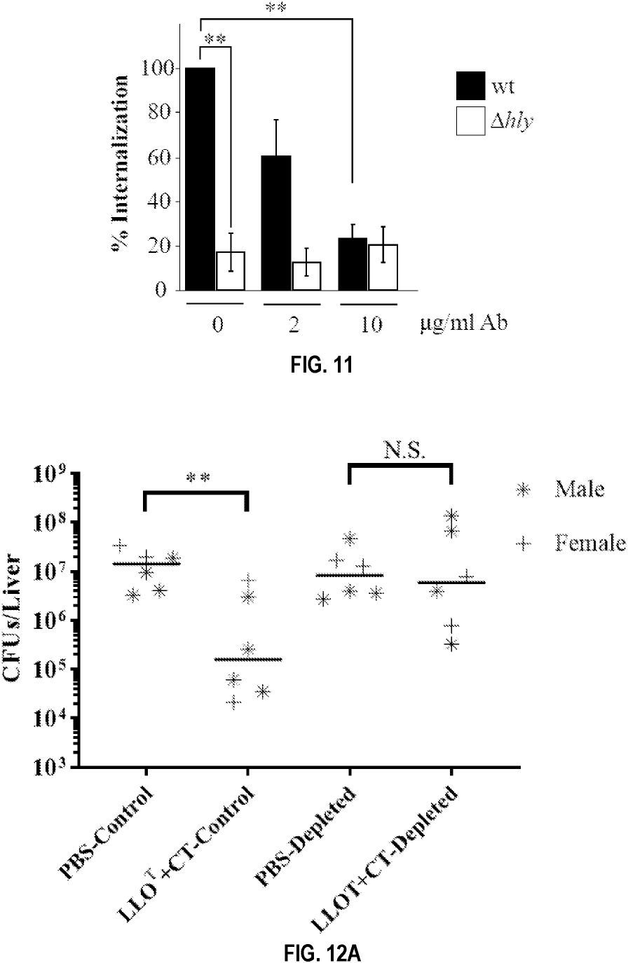

Adjuvants were introduced in the present vaccine design. Key players that mediate sterilizing adaptive immune response to L. monocytogenes include CD4⁺ Th1 cells producing IFN-y, which are known to activate the bactericidal activity of macrophages and CD8⁺ cytotoxic T cell responses. Studies by Edelson et al. using a murine infection model suggested that, unlike the robust T cell responses, B cell responses and the production of antibodies were limited in response to L. monocytogenes infection. However, the adoptive transfer of monoclonal LLO-neutralizing antibodies, but not of anti-LLO non-neutralizing antibodies, protected naïve mice against sub-lethal and lethal doses of L. monocytogenes. The protective effect was attributed to the neutralization of LLO within the phagosomes of infected cells. LLO-neutralizing antibodies can in addition abrogate the extracellular activities of LLO, as evidenced by their ability to inhibit LLO-mediated bacterial internalization into hepatocytes (FIG. 11). These observations indicate that the production of LLO-neutralizing antibodies can promote -in addition to the T cell protective response- protection against the pathogen.

Cholera toxin, an experimental adjuvant, was used herein for eliciting balanced and robust T and B cells immune responses. Inoculation of LLO^{T} plus cholera toxin significantly protected mice against L. monocytogenes (FIGS. 4A-4B). The combination of cholera toxin with LLO^{T} significantly increased the production of LLO-specific IgG1 and IgG2a isotypes (FIG. 6), with IgG2a isotype class switching being known to be driven by IFN-y. Importantly, these antibodies neutralized LLO activity (FIG. 7). Also, a pronounced Th1 response characterized by the production of IFN-y was observed (FIGS. 8A-8E). To interrogate the role of LLO-neutralizing antibodies and tease apart their contribution from the role of Th1 protective response, Alum was used to elicit robust antibody production without concurrently inducing strong Th1 T cell responses. Inoculation of Alum and LLO^{T} did not result in reduced infectious burden (FIGS. 5A-5B). Alum was less efficient than cholera toxin in eliciting LLO-specific total IgG and IgG1. In addition, the levels of IgG2a, IgG2b, and IgG3 were substantially lower when using alum in comparison to cholera toxin (FIG. 6). The IgG2a, IgG2b, and IgG3 isotypes are thought to be the chief complement-fixing and opsonizing isotypes in mice. Most importantly, IgGs from mice treated with LLO^{T} + Alum did not neutralize LLO as observed in mice treated with LLO^{T} + CT.

The ability of the two adjuvants to elicit LLO-specific T helper responses was compared. The major distinction of the LLO^{T}+ CT treatment is the significant increase in IFN-y⁺ CD4⁺ T cells, indicating a Th1 dominated response. This shows the critical role of IFN-y in the activation of CD8⁺ T cells and macrophages for the clearance of L. monocytogenes. IFN-y is critical for Ig class switching to the IgG2a isotype, with the associated Th1 response important for IgG2b and IgG3 production. To determine if the Th1 response, which is implicated in protection, is sufficient in the absence of LLO-specific antibodies, µMT^{-/-} mice that lack mature B cells were immunized. This experiment led to two major conclusions. First, MT^{-/-} mice are more resistant to L. monocytogenes infection as shown by the substantial decrease in CFU recovered from spleen and liver in comparison to WT mice. Indeed, L. monocytogenes was shown to stimulate IL-10 producing B cells leading to decreased macrophage anti-listeria responses. Similar observations were reported with SCID mice, which are deficient in both T and B cells, infected with L. monocytogenes despite the acknowledged protective role of T cells. Second, there was a significant reduction in bacteria CFUs in the livers and spleens of µMT^{-/-} mice immunized with LLO^{T}+CT compared to all other groups of animals (FIGS. 9A-9B).

### Example 9 Effective Immunization with LLOT Plus Cholera Toxin is Mediated by T Cells

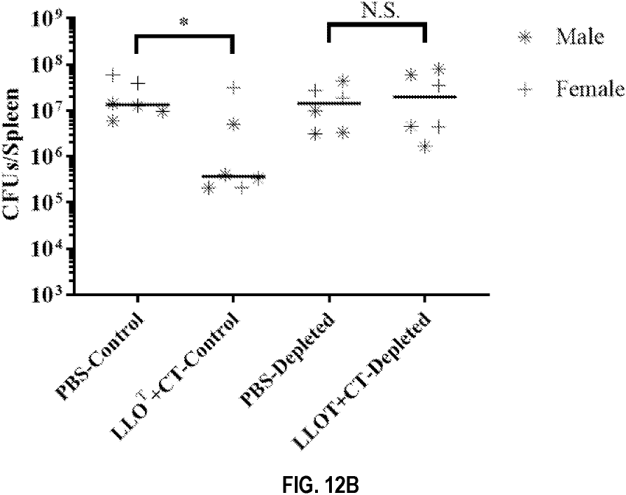

In order to confirm the role of T cells in the anti-L. monocytogenes protection of mice immunized with LLOT + CT, T cells were depleted after immunization by administering a cocktail of CD8- and CD4-cell-depleting antibodies, or control isotypes, 48 h before and 24 h after infection. Analysis of circulating leukocytes confirmed the efficacy of T cell depletion, whereas B cells, natural killer cells, and dendritic cells remained unaffected. When isotype control antibodies were administered to mice immunized with LLOT + CT, significant decreases were observed in bacterial burden 72 h post-infection (FIGS. 12A-12B). Importantly, T cell depletion post-immunization abrogated protection in the LLOT + CT group, demonstrating that T cells are required for effective immunization (FIGS. 12A-12B).

### References

1 Vazquez-Boland, J. A., Dominguez-Bernal, G., Gonzalez-Zorn, B., Kreft, J. & Goebel, W. Pathogenicity islands and virulence evolution in Listeria. Microbes Infect 3, 571-584 (2001).

2 Teberg, A. J., Yonekura, M. L., Salminen, C. & Pavlova, Z. Clinical manifestations of epidemic neonatal listeriosis. Pediatr Infect Dis J6, 817-820 (1987).

3 Mylonakis, E., Paliou, M., Hohmann, E. L., Calderwood, S. B. & Wing, E. J. Listeriosis during pregnancy: a case series and review of 222 cases. Medicine (Baltimore) 81, 260-269 (2002).

4 Robbins, J. R. & Bakardjiev, A. I. Pathogens and the placental fortress. Curr Opin Microbiol 15, 36-43, doi:10.1016/j.mib.2011.11.006 (2012).

5 Berche, P. Bacteremia is required for invasion of the murine central nervous system by Listeria monocytogenes. Microb Pathog 18, 323-336, doi:10.1006/mpat.1995.0029 (1995).

6 Disson, O. & Lecuit, M. Targeting of the central nervous system by Listeria monocytogenes. Virulence 3, 213-221, doi:10.4161/viru.19586 (2012).

7 Scharff, R. L. Economic burden from health losses due to foodborne illness in the United States. J Food Prot 75, 123-131, doi:10.4315/0362-028X.JFP-11-058 (2012).

8 Center for Disease Control and Prevention (CDC). National Listeria surveillance annual Summary, 2013. Atlanta, Georgia: US Department of Health and Human Services, CDC, 2015.

9 Pohl, A. M. et al. Differences Among Incidence Rates of Invasive Listeriosis in the U.S. FoodNet Population by Age, Sex, Race/Ethnicity, and Pregnancy Status, 2008-2016. Foodborne Pathog Dis, doi:10.1089/fpd.2018.2548 (2019).

10 Clark, D. R. et al. Perinatal Listeria monocytogenes susceptibility despite preconceptual priming and maintenance of pathogen-specific CD8(+) T cells during pregnancy. Cell Mol Immunol 11, 595-605, doi:10.1038/cmi.2014.84 (2014).

11 Dowd, G. C. et al. Listeria monocytogenes mutants defective in gallbladder replication represent safety-enhanced vaccine delivery platforms. Hum Vaccin Immunother 12, 2059-2063, doi:10.1080/21645515.2016.1154248 (2016).

12 McLaughlin, H. P., Bahey-El-Din, M., Casey, P. G., Hill, C. & Gahan, C. G. A mutant in the Listeria monocytogenes Fur-regulated virulence locus (frvA) induces cellular immunity and confers protection against listeriosis in mice. J Med Microbiol 62, 185-190, doi:10.1099/jmm.0.049114-0 (2013).

13 Mackaness, G. B. Cellular resistance to infection. J Exp Med 116, 381-406 (1962).

14 Le, D. T., Dubenksy, T. W., Jr. & Brockstedt, D. G. Clinical development of Listeria monocytogenes-based immunotherapies. Semin Oncol 39, 311-322, doi:10.1053/j.seminoncol.2012.02.008 (2012).

15 Fares, E. et al. Vaccine strain Listeria monocytogenes bacteremia occurring 31 months after immunization. Infection, doi:10.1007/s15010-018-1249-7 (2018).

16 Calderon-Gonzalez, R. et al. Cellular vaccines in listeriosis: role of the Listeria antigen GAPDH. Front Cell Infect Microbiol 4, 22, doi:10.3389/fcimb.2014.00022 (2014).

17 Jensen, S. et al. Adenovirus-based vaccine against Listeria monocytogenes: extending the concept of invariant chain linkage. J Immunol 191, 4152-4164, doi:10.4049/jimmunol.1301290 (2013).

18 Rodriguez-Del Rio, E. et al. A gold glyco-nanoparticle carrying a Listeriolysin O peptide and formulated with Advax delta inulin adjuvant induces robust T-cell protection against listeria infection. Vaccine 33, 1465-1473, doi:10.1016/j.vaccine.2015.01.062 (2015).

19 Luo, X. & Cai, X. A combined use of autolysin p60 and listeriolysin O antigens induces high protective immune responses against Listeria monocytogenes infection. Curr Microbiol 65, 813-818, doi:10.1007/s00284-012-0238-9 (2012).

20 Gaillard, J. L., Berche, P. & Sansonetti, P. Transposon mutagenesis as a tool to study the role of hemolysin in the virulence of Listeria monocytogenes. Infect Immun 52, 50-55 (1986).

21 Geginat, G., Schenk, S., Skoberne, M., Goebel, W. & Hof, H. A novel approach of direct ex vivo epitope mapping identifies dominant and subdominant CD4 and CD8 T cell epitopes from Listeria monocytogenes. J Immunol 166, 1877-1884 (2001).

22 Hernandez-Flores, K. G. & Vivanco-Cid, H. Biological effects of listeriolysin O: implications for vaccination. Biomed Res Int 2015, 360741, doi:10.1155/2015/360741 (2015).

23 Tweten, R. K. Cholesterol-dependent cytolysins, a family of versatile pore-forming toxins. Infect Immun 73, 6199-6209, doi:10.1128/IAI.73.10.6199-6209.2005 (2005).

24 Farrand, A. J., LaChapelle, S., Hotze, E. M., Johnson, A. E. & Tweten, R. K. Only two amino acids are essential for cytolytic toxin recognition of cholesterol at the membrane surface. Proc Natl Acad Sci USA 107, 4341-4346, doi:10.1073/pnas.0911581107 (2010).

25 Vadia, S. et al. The pore-forming toxin listeriolysin O mediates a novel entry pathway of L. monocytogenes into human hepatocytes. PLoS Pathog 7, e1002356, doi:10.1371/journal.ppat.1002356 (2011).

26 Michel, E., Reich, K. A., Favier, R., Berche, P. & Cossart, P. Attenuated mutants of the intracellular bacterium Listeria monocytogenes obtained by single amino acid substitutions in listeriolysin O. Mol Microbiol 4, 2167-2178 (1990).

27 Kohda, C. et al. Dissociated linkage of cytokine-inducing activity and cytotoxicity to different domains of listeriolysin O from Listeria monocytogenes. Infect Immun 70, 1334-1341 (2002).

28 Lam, J. G. T. et al. Host cell perforation by listeriolysin O (LLO) activates a Ca(2+)-dependent cPKC/Rac1/Arp2/3 signaling pathway that promotes Listeria monocytogenes internalization independently of membrane resealing. Mol Biol Cell 29, 270-284, doi:10.1091/mbc.E17-09-0561 (2018).

29 Malyala, P. & Singh, M. Endotoxin limits in formulations for preclinical research. J Pharm Sci 97, 2041-2044, doi:10.1002/jps.21152 (2008).

30 Kitamura, D., Roes, J., Kuhn, R. & Rajewsky, K. A B cell-deficient mouse by targeted disruption of the membrane exon of the immunoglobulin mu chain gene. Nature 350, 423-426, doi:10.1038/350423a0 (1991).

31 Mattsson, J. et al. Cholera toxin adjuvant promotes a balanced Th1/Th2/Th17 response independently of IL-12 and IL-17 by acting on Gsalpha in CD11b(+) DCs. Mucosal Immunol 8, 815-827, doi:10.1038/mi.2014.111 (2015).

32 Edelson, B. T., Cossart, P. & Unanue, E. R. Cutting edge: paradigm revisited: antibody provides resistance to Listeria infection. J Immunol 163, 4087-4090 (1999).

33 Bonnegarde-Bernard, A. et al. IKKbeta in intestinal epithelial cells regulates allergen-specific IgA and allergic inflammation at distant mucosal sites. Mucosal Immunol 7, 257-267, doi:10.1038/mi.2013.43 (2014).

34 Oleszycka, E. & Lavelle, E. C. Immunomodulatory properties of the vaccine adjuvant alum. Curr Opin Immunol 28, 1-5, doi:10.1016/j.coi.2013.12.007 (2014).

35 Bhardwaj, V., Kanagawa, O., Swanson, P. E. & Unanue, E. R. Chronic Listeria infection in SCID mice: requirements for the carrier state and the dual role of T cells in transferring protection or suppression. J Immunol 160, 376-384 (1998).

36 Lane, F. C. & Unanue, E. R. Requirement of thymus (T) lymphocytes for resistance to listeriosis. J Exp Med 135, 1104-1112 (1972).

37 Miki, K. & Mackaness, G. B. The Passive Transfer of Acquired Resistance to Listeria Monocytogenes. J Exp Med 120, 93-103 (1964).

38 Christie, M. P., Johnstone, B. A., Tweten, R. K., Parker, M. W. & Morton, C. J. Cholesterol-dependent cytolysins: from water-soluble state to membrane pore. Biophys Rev 10, 1337-1348, doi:10.1007/s12551-018-0448-x (2018).

39 Darji, A. et al. Neutralizing monoclonal antibodies against listeriolysin: mapping of epitopes involved in pore formation. Infect Immun 64, 2356-2358 (1996).

40 Sun, R. & Liu, Y. Listeriolysin O as a strong immunogenic molecule for the development of new anti-tumor vaccines. Hum Vaccin Immunother 9, 1058-1068, doi:10.4161/hv.23871 (2013).

41 Snapper, C. M. & Paul, W. E. Interferon-gamma and B cell stimulatory factor-1 reciprocally regulate Ig isotype production. Science 236, 944-947 (1987).

42 Michaelsen, T. E., Kolberg, J., Aase, A., Herstad, T. K. & Hoiby, E. A. The four mouse IgG isotypes differ extensively in bactericidal and opsonophagocytic activity when reacting with the P1.16 epitope on the outer membrane PorA protein of Neisseria meningitidis. Scand J Immunol 59, 34-39 (2004).

43 Germann, T. et al. Interleukin-12 profoundly up-regulates the synthesis of antigen-specific complement-fixing IgG2a, IgG2b and IgG3 antibody subclasses in vivo. Eur J Immunol 25, 823-829, doi:10.1002/eji.1830250329 (1995).

44 Calderon-Gonzalez, R. et al. GNP-GAPDH1-22 nanovaccines prevent neonatal listeriosis by blocking microglial apoptosis and bacterial dissemination. Oncotarget 8, 53916-53934, doi:10.18632/oncotarget.19405 (2017).

45 Calderon-Gonzalez, R. et al. Pregnancy Vaccination with Gold Glyco-Nanoparticles Carrying Listeria monocytogenes Peptides Protects against Listeriosis and Brain- and Cutaneous-Associated Morbidities. Nanomaterials (Basel) 6, doi:10.3390/nano6080151 (2016).

46 Simister, N. E. Placental transport of immunoglobulin G. Vaccine 21, 3365-3369 (2003).

47 Palmeira, P., Quinello, C., Silveira-Lessa, A. L., Zago, C. A. & Carneiro-Sampaio, M. IgG placental transfer in healthy and pathological pregnancies. Clin Dev Immunol 2012, 985646, doi:10.1155/2012/985646 (2012).

48 Kim, J. et al. FcRn in the yolk sac endoderm of mouse is required for IgG transport to fetus. J Immunol 182, 2583-2589, doi:10.4049/jimmunol.0803247 (2009).

49 Cowan, G. J., Atkins, H. S., Johnson, L. K., Titball, R. W. & Mitchell, T. J. Immunisation with anthrolysin O or a genetic toxoid protects against challenge with the toxin but not against Bacillus anthracis. Vaccine 25, 7197-7205, doi:10.1016/j.vaccine.2007.07.040 (2007).

50 Paton, J. C. et al. Purification and immunogenicity of genetically obtained pneumolysin toxoids and their conjugation to Streptococcus pneumoniae type 19F polysaccharide. Infect Immun 59, 2297-2304 (1991).

51 Jost, B. H., Trinh, H. T., Songer, J. G. & Billington, S. J. Immunization with genetic toxoids of the Arcanobacterium pyogenes cholesterol-dependent cytolysin, pyolysin, protects mice against infection. Infect Immun 71, 2966-2969 (2003).

52 Alexander, J. E. et al. Immunization of mice with pneumolysin toxoid confers a significant degree of protection against at least nine serotypes of Streptococcus pneumoniae. Infect Immun 62, 5683-5688 (1994).

53 Bubeck Wardenburg, J. & Schneewind, O. Vaccine protection against Staphylococcus aureus pneumonia. J Exp Med 205, 287-294, doi:10.1084/j em.20072208 (2008).

54 Hernandez-Flores, K. G. et al. Evaluation of the safety and adjuvant effect of a detoxified listeriolysin O mutant on the humoral response to dengue virus antigens. Clin Exp Immunol 188, 109-126, doi:10.1111/cei.12906 (2017).

55 Wallecha, A. et al. Listeria monocytogenes-derived listeriolysin O has pathogen-associated molecular pattern-like properties independent of its hemolytic ability. Clin Vaccine Immunol 20, 77-84, doi:10.1128/CVI.00488-12 (2013).

### SEQUENCES

SEQ ID NO: 1, AMINO ACID SEQUENCE WILD TYPE LLO:

MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIW
EKKKKSINQNNADIQWNAISSLTYPGALVKANSELVENQPDVLPVKRDSL
TLSIDLPGMTNQDNKIWKNATKSNVNNAVNTLVERWNEKYAQAYPNVSAK
IDYDDEMAYSESQLIAKFGTAFKAVNNSLNVNFGAISEGKMQEEVISFKQ
IYYNVNVNEPTRPSRFFGKAVTKEQLQALGVNAENPPAYISSVAYGRQVY
LKLSTNSHSTKVKAAFDAAVSGKSVSGDVELTNIIKNSSFKAVIYGGSAK
DEVQIIDGNLGDLRDILKKGATFNRETPGVPIAYTTNFLKDNELAVIKNN
SEYIETTSKAYTDGKINIDHSGGYVAQFNISWDEVNYDPEGNEIVQHKNW
SENNKSKLAHFTSSIYLPGNARNINVYAKECTGLAWEWWRTVIDDRNLPL
VKNRNISIWGTTLYPKYSNKVDNPIE

SEQ ID NO: 2, SEQUENCE OF LLO-ENCODING GENE:

1 - atg aaa aaa ata atg cta gtt ttt att aca
31 - ctt ata tta gtt agt cta cca att gcg caa
61 - caa act gaa gca aag gat gca tct gca ttc
91 - aat aaa gaa aat tca att tca tcc atg gca
121 - cca cca gca tct ccg cct gca agt cct aag
151 - acg cca atc gaa aag aaa cac gcg gat gaa
181 - atc gat aag tat ata caa gga ttg gat tac
211 - aat aaa aac aat gta tta gta tac cac gga
241 - gat gca gtg aca aat gtg ccg cca aga aaa
271 - ggt tac aaa gat gga aat gaa tat att gtt
301 - gtg gag aaa aag aag aaa tcc atc aat caa
331 - aat aat gca gac att caa gtt gtg aat gca
361 - att tcg agc cta acc tat cca ggt gct ctc
391 - gta aaa gcg aat tcg gaa tta gta gaa aat
421 - caa cca gat gtt ctc cct gta aaa cgt gat
451 - tca tta aca ctc agc att gat ttg cca ggt
481 - atg act aat caa gac aat aaa atc gtt gta
511 - aaa aat gcc act aaa tca aac gtt aac aac
541 - gca gta aat aca tta gtg gaa aga tgg aat
571 - gaa aaa tat gct caa gct tat cca aat gta
601 - agt gca aaa att gat tat gat gac gaa atg
631 - gct tac agt gaa tca caa tta att gcg aaa
661 - ttt ggt aca gca ttt aaa gct gta aat aat
691 - agc ttg aat gta aac ttc ggc gca atc agt
721 - gaa ggg aaa atg caa gaa gaa gtc att agt
751 - ttt aaa caa att tac tat aac gtg aat gtt
781 - aat gaa cct aca aga cct tcc aga ttt ttc
811 - ggc aaa gct gtt act aaa gag cag ttg caa
841 - gcg ctt gga gtg aat gca gaa aat cct cct
871 - gca tat atc tca agt gtg gcg tat ggc cgt
901 - caa gtt tat ttg aaa tta tca act aat tcc
931 - cat agt act aaa gta aaa gct gct ttt gat
961 - gct gcc gta agc gga aaa tct gtc tca ggt
991 - gat gta gaa cta aca aat atc atc aaa aat
1021 - tct tcc ttc aaa gcc gta att tac gga ggt
1051 - tcc gca aaa gat gaa gtt caa atc atc gac
1081 - ggc aac ctc gga gac tta cgc gat att ttg
1111 - aaa aaa ggc gct act ttt aat cga gaa aca
1141 - cca gga gtt ccc att gct tat aca aca aac
1171 - ttc cta aaa gac aat gaa tta gct gtt att
1201 - aaa aac aac tca gaa tat att gaa aca act
1231 - tca aaa gct tat aca gat gga aaa att aac
1261 - atc gat cac tct gga gga tac gtt gct caa
1291 - ttc aac att tct tgg gat gaa gta aat tat
1321 - gat cct gaa ggt aac gaa att gtt caa cat
1351 - aaa aac tgg agc gaa aac aat aaa agc aag
1381 - cta gct cat ttc aca tcg tcc atc tat ttg
1411 - cca ggt aac gcg aga aat att aat gtt tac
1441 - gct aaa gaa tgc act ggt tta gct tgg gaa
1471 - tgg tgg aga acg gta att gat gac cgg aac
1501 - tta cca ctt gtg aaa aat aga aat atc tcc
1531 - atc tgg ggc acc acg ctt tat ccg aaa tat
1561 - agt aat aaa gta gat aat cca atc gaa taa

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

2. The polypeptide of claim 1, wherein the amino acid substitution is at amino acid position 515.

3. The polypeptide of claim 1, wherein the amino acid substitution is at amino acid position 516.

4. The polypeptide of claim 1, wherein the non-toxic listeriolysin O comprises amino acid substitutions at amino acid positions 515 and 516.

5. The polypeptide of claim 1,wherein the amino acid substitution at amino acid position 515 is selected from the group consisting ofT515G and T515A.

6. The polypeptide of claim 1, wherein the amino acid substitution at amino acid position 516 is selected from the group consisting of L516G and L516A.

7. The polypeptide of claim 1,wherein the non-toxic listeriolysin O binds to a cell membrane.

8. The polypeptide of claim 1, wherein the non-toxic listeriolysin O binds to an antigen-presenting cell.

9. A nucleic acid comprising: a genetically modified listeriolysin O gene comprising one or more point mutations, wherein the genetically modified listeriolysin O gene encodes a polypeptide of claim 1.

10. A recombinant DNA vector comprising the nucleic acid of claim 9.

11. A vaccine comprising: a non-toxic listeriolysin O comprising an amino acid substitution at one or more amino acid positions when compared to SEQ ID NO: 1, wherein the one or more amino acid positions are selected from the group consisting of 515 and 516.

12-18. (canceled)

19. The vaccine of claim 11, further comprising one or more adjuvants.

20. The vaccine of claim 19, wherein the one or more adjuvants is cholera toxin.

21. A method of preventing a Listeria infection, comprising administering to a subject an effective amount of the vaccine of claim 11.

22. The method of claim 21, wherein administering the vaccine activates a CD4+ Th1, a CD8+ T cell, or a B cell.

23. A method of preventing a Listeria infection, comprising administering to a subject an effective amount of a vaccine comprising:
a polypeptide comprising: a non-toxic listeriolysin O comprising an amino acid substitution at one or more amino acid positions when compared to SEQ ID NO: 1, wherein the one or more amino acid positions are selected from the group consisting of 515 and 516.

24-30. (canceled)

31. The method of claim 23,further comprising one or more adjuvants.

32. The method of claim 31, wherein the one or more adjuvants is cholera toxin.

33. (canceled)

34. The method of claim 23,wherein the Listeria is Listeria Monocytogenes.

35. The method of claim 23, wherein the subject is a human.
